(19) United States
(12) Patent Application Publication
(10) Pub. No.: US 2024/0219333 A1
(43) Pub. Date: Jul. 4, 2024

(54) IMPEDANCE-BASED INTESTINAL ORGANOID EVALUATION SYSTEM

(71) Applicants: KOREA RESEARCH INSTITUTE OF CHEMICAL TECHNOLOGY, Daejeon (KR); KOREA RESEARCH INSTITUTE OF BIOSCIENCE AND BIOTECHNOLOGY, Daejeon (KR)

(72) Inventors: Jung-Hwa Oh, Daejeon (KR); Jaehwan Ahn, Daejeon (KR); Seokjoo Yoon, Daejeon (KR); Hyoung-Yun Han, Daejeon (KR); Mi Young Son, Daejeon (KR); Kwang Bo Jung, Daejeon (KR); Cho Rok Jung, Daejeon (KR); Janghwan Kim, Daejeon (KR)

(21) Appl. No.: 18/003,757

(22) PCT Filed: Jun. 15, 2021
(86) PCT No.: PCT/KR2021/007470

§ 371 Date: Jul. 14, 2023

(30) Foreign Application Priority Data
Jul. 30, 2020 (KR) 10-2020-0095263

(51) Int. Cl. G01N 27/04; C12M 1/26; C12M 1/34; G01N 33/483

(52) U.S. Cl. CPC G01N 27/041; C12M 33/04; C12M 33/12; C12M 41/40; G01N 33/4833

(57) ABSTRACT

The present invention provides an impedance-based organoid evaluation system comprising an organoid deformation generating unit including a first tube and a plurality of second tubes having a diameter smaller than that of the first tube, connected to one end of the first tube or inserted therein; and an impedance measuring unit connected to the organoid deformation generating unit and including an impedance analyzer for measuring the impedance of the organoid, and evaluating the organoid from the impedance measured by the impedance measuring unit.

### BACKGROUND OF THE INVENTION

### 1. Field of the Invention

The present invention relates to an impedance measurement system capable of evaluating the maturity and barrier integrity of a three-dimensional intrinsic organoid among human organoid models (human intestinal organoid, HIO), and a method for measuring the degree of maturation of an intestinal organoid and whether the barrier integrity is damaged by changes in impedance.

### 2. Description of the Related Art

In the intestinal tract, the intestinal mucosa performs an immunological function of blocking the inflow of microorganisms, antigens, and toxins into the bloodstream during digestion and absorption. The intestinal mucosa cells, which act as the primary barrier, maintain a certain gap between cells with a single cell layer, and when any stimulus or damage is applied, the permeability increases so that various polymer substances can pass through the gap between the cells. Antigens that enter the blood through this cause a serious immune response and cause various chronic immune diseases.

Studies on the intestinal organoid simulating such an intestinal structure are being actively conducted, and a technique for evaluating the membrane function or barrier integrity of the intestinal organoid is being studied.

As a representative example, in the case of transepithelial/transendothelial electrical resistance (TEER), there is a method of measuring the change in resistance of current flowing between cells by incubating cells in a porous structure, placing them slightly away from the bottom, and immersing them in a culture medium with an electrode. However, in the case of an intestinal organoid, it is a three-dimensional structure and is formed through differentiation and maturation. The intestinal organoid that has undergone this process of maturation forms a more complex three-dimensional structure with unevenly folding in appearance, so it is difficult to evaluate the degree of membrane damage and barrier integrity of the intestinal organoid with the conventional TEER measurement technology.

To compensate for this, there is a method of measuring extracellular resistance by forming a single-microchannel smaller than cell tissue, placing the cell tissue inside, filling the culture medium, and flowing alternating current. However, the method can measure only a spherical three-dimensional structure with a uniform and full interior. The intestinal organoid is a hollow balloon-shaped structure that is more uneven in appearance as it matures, making it difficult to place in a small microchannel, and current flows outside the area touched by the uneven appearance. Also, because the intestinal organoid is a soft structure, it is often torn when passing through a narrow opening.

### SUMMARY OF THE INVENTION

In one aspect, an object of the present invention is to provide an impedance-based measurement system that can evaluate the barrier integrity of an empty, non-uniform structure without deviation of data. A stem cell-derived intestinal organoid shows a structural complexity similar to the biological tissue, such as increasing maturity-related gene expression and increasing size and number of budding structures, so it is very important to evaluate them without any deviation. In particular, the intestinal organoid is very useful in that it enables real-time monitoring of the barrier integrity of the organoid in a simple and non-invasive way. The intestinal organoid can be used as a test system for the development of a therapeutic agent for intestinal diseases such as Crohn's disease, and can also be used as a model for evaluating the efficacy of the intestinal microbiome. Therefore, the impedance technique that can evaluate the barrier integrity of the intestinal organoid can also be used for drug, chemical, toxin and microbial screening and efficacy studies that may affect the barrier integrity of the intestines. Furthermore, it can be used to evaluate the degree of membrane damage and barrier integrity of the complicated three-dimensional organoid structures that mimic various living tissues such as intestinal organoids as well as liver and lung organoids.

To achieve the above object, in one aspect of the present invention, the present invention provides an impedance-based intestinal organoid evaluation system comprising:
an intestinal organoid deformation generating unit including a first tube; and at least three second tubes having a diameter smaller than that of the first tube, and being connected to one end of the first tube or inserted therein; and
an impedance measuring unit including an impedance analyzer for measuring an impedance of an intestinal organoid, the impedance analyzer being connected to the intestinal organoid deformation generating unit; and
wherein the impedance-based intestinal organoid evaluation system is configured to evaluate the intestinal organoid from the impedance measured by the impedance measuring unit.

In another aspect of the present invention, the present invention provides a method for evaluating the barrier integrity of an organoid comprising the following steps:
step of filling the inside of the first tube of the impedance-based organoid evaluation system with physiological saline or culture medium, and introducing the organoid into the first tube;
step of positioning the organoid to contact one end of the second tube, and measuring the first impedance using an impedance analyzer;
step of forming a negative pressure in the organoid deformation generating unit, and measuring the second impedance; and
step of evaluating the barrier integrity of the organoid from the first impedance and the second impedance.

In another aspect of the present invention, the present invention provides a method for evaluating the barrier integrity of an organoid comprising the following steps:
step of filling the inside of the first tube of the impedance-based organoid evaluation system with a substance that induces membrane function damage and efficacy, and injecting the organoid into the first tube;
step of positioning the organoid to contact one end of the second tube, and measuring the first impedance using an impedance analyzer;
step of forming a negative pressure in the organoid deformation generating unit, and measuring the second impedance;
step of measuring the third impedance at regular time intervals in a state where the negative pressure is stable; and
step of evaluating the barrier integrity affected by the substance that induces membrane function damage and efficacy from the first impedance, the second impedance and the third impedance.

### Advantageous Effect

The impedance-based organoid evaluation system provided in one aspect of the present invention can evaluate the barrier integrity such as the degree of membrane damage without deviation of data on the membrane characteristics of a hollow and uneven structure.

In addition, the method for evaluating the barrier integrity of an organoid provided in one aspect of the present invention is highly useful in that it is possible to monitor the degree of membrane damage in real time in a very simple and non-invasive way.

### BRIEF DESCRIPTION OF THE DRAWINGS

FIG. 1 is a schematic diagram showing an example of the impedance-based organoid evaluation system;

FIG. 2 is a schematic diagram showing an example of the impedance-based organoid evaluation system;

FIG. 3 is a schematic diagram showing a three-dimensional intestinal organoid evaluation impedance device;

FIG. 4 is a set of images and graphs showing the increase of the barrier integrity according to the maturation of the intestinal organoid;

FIG. 5 is a set of graphs showing the change of the impedance resistance value between the immature control group and the mature intestinal organoid; and

FIG. 6 is a graph showing the change of the resistance value according to the presence or absence of 1× trypsin (barrier integrity impairment inducing substance) after the intestinal organoid is adsorbed to the deformation generating unit (200 mV, 50 Hz, AC, Red; with trypsin, Black: without trypsin, the black dotted line indicates the time when the resistance value change is 50% (t_{50%})).

### DESCRIPTION OF THE PREFERRED EMBODIMENTS

Hereinafter, the present invention is described in detail.

In one aspect of the present invention, the present invention provides an impedance-based intestinal organoid evaluation system comprising:
an intestinal organoid deformation generating unit including a first tube; and at least three second tubes having a diameter smaller than that of the first tube, and being connected to one end of the first tube or inserted therein; and
an impedance measuring unit including an impedance analyzer for measuring an impedance of an intestinal organoid, the impedance analyzer being connected to the intestinal organoid deformation generating unit; and
wherein the impedance-based intestinal organoid evaluation system is configured to evaluate the intestinal organoid from the impedance measured by the impedance measuring unit.

The advantage of this impedance-based organoid evaluation system is that there is less variation in the physical properties of the intestinal organoid compared to a single channel. In the case of a single channel, when adsorbing non-uniform cell tissue, the degree of suction into the channel varies depending on the region even at the same negative pressure. Since the degree of suction into the channel is directly related to the resistance value, it is important to keep the degree constant. In the present invention, the degree of suction is constantly maintained by dispersing the partial pressure applied to the organoid through a multi-channel rather than a single channel. This effect can be increased by increasing the number of multi-channels.

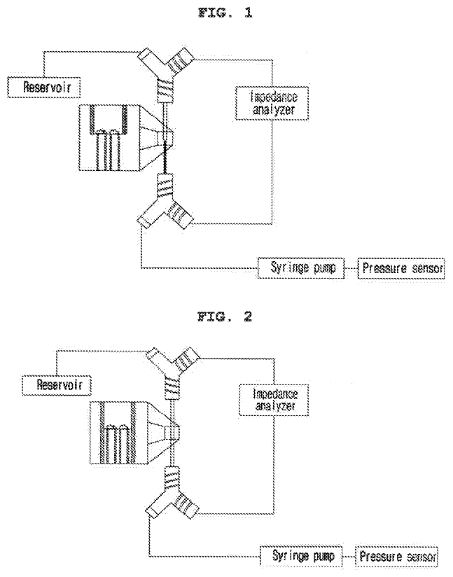

FIGS. 1 to 3 are schematic diagrams showing an example of the impedance-based organoid evaluation system provided in one aspect of the present invention.

The impedance-based organoid evaluation system provided in one aspect of the present invention comprises an organoid deformation generating unit including a first tube and a plurality of second tubes having a diameter smaller than that of the first tube and connected to one end of the first tube or inserted therein.

The first tube may have a hollow cylindrical shape, or a cylindrical shape. The second tube may also have a hollow cylindrical shape, or a cylindrical shape.

The first tube may constitute a primary channel as a single channel, and the second tube may constitute a secondary multi-channel as a multi-channel.

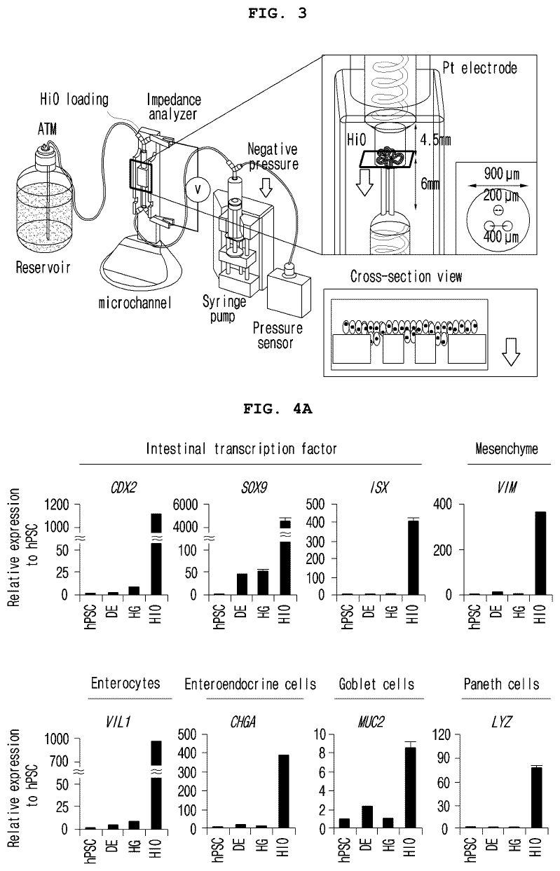

The plurality of second tubes can be connected to one end of the first tube as shown in FIG. 1, and can be inserted and installed therein as shown in FIG. 2. When the second tube is connected to one end of the first tube, the second tube can be connected to a single channel of the first tube to form a multi-channel, and in this case, the end part at which the first tube and the second tube are connected can be sealed except for the channel portion of the second tube. In addition, when the second tube is inserted and installed inside the first tube, the first tube is longer than the second tube, and a plurality of second tubes can be modularized and mounted inside the first tube.

The diameter of the second tube preferably has a diameter of 51 to 30 of the diameter of the first tube, and can have a diameter of 10% to 28%, 15% to 25%, and 20% to 24% of the diameter of the first tube. In addition, the second tube is preferably at least three or more, through this, it is possible to configure a multi-channel. By configuring such a multi-channel, the change in the physical properties of the organoid is remarkably small, and the degree of suction into the channel is constant and maintained even at a constant negative pressure, so that the maturity and barrier integrity of the organoid can be accurately evaluated.

In addition, the organoid deformation generating unit comprises a third tube connected to the other end of the first tube; and a fourth tube connected to one end of the first tube or one end of the plurality of second tubes.

The third tube can have a Y-shape, and the Y-shaped third tube can include a tube i connected to one end of the first tube; a tube u connected to the reservoir to put the material in the reservoir into the first tube; and a tube iii including an electrode material.

The reservoir can store one of a physiological saline, a culture solution, a membrane function impairment inducing solution, and a membrane function efficacy inducing material.

The electrode material placed inside the pipe iii can be in the form of a coil-shaped wire, and can be formed to extend into the tube i.

The fourth tube has a Y-shape, and the Y-shaped fourth tube includes a tube I connected to one end of the first tube or one end of the plurality of second tubes; a tube II connected to a syringe pump; and a tube III including an electrode material.

The syringe pump can be connected to a pressure sensor.

The electrode material placed inside the tube III can be in the form of a coil-shaped wire, and can be formed to extend into the tube I.

For example, the coil-shaped gold (Au) wire placed inside the tube i can be extended into the tube i, and the coil-shaped platinum (Pt) wire placed inside the tube III can be extended into the tube I. When the gold and platinum wires are extended in this way, the gold and platinum wires can more easily share the materials filled in the first, second, third, and fourth tubes, so that the impedance value change can be measured accurately and reliably.

The impedance-based organoid evaluation system provided in one aspect of the present invention comprises an impedance measuring unit connected to the organoid deformation generating unit and including an impedance analyzer for measuring the impedance of the organoid. The impedance analyzer can include a working electrode and a counter electrode, and can be connected to the organoid deformation generating unit. As a specific example, the working electrode of the impedance analyzer can be connected to the electrode material of the tube iii including the electrode material of the organoid deformation generating unit, and preferably, the electrode material can be composed of a coil-shaped gold wire and connected to a gold wire protruding to the outside. In addition, the counter electrode of the impedance analyzer can be connected to the electrode material of the tube III including the electrode material of the organoid deformation generating unit, and preferably, the electrode material can be composed of a coil-shaped platinum wire and connected to a platinum wire protruding to the outside.

As a specific example, the impedance-based organoid evaluation system can be composed of a first tube that forms a primary channel approximately the diameter of the intestinal organoid (˜900 μm) and second tubes constituting multi-channels having at least three or more and having a diameter of about 200 μm. The first and second tubes are vertically constructed and the inside is filled with a culture medium or physiological saline, and both ends of the channel (one end of the first tube and one end of the second tube) can be connected to the Y-shaped tubes (third tube and forth tube), respectively. The intestinal organoid enters the upper part of the first tube through the upper part entrance, and the entrance is sealed again. The lower part of the second tubes is connected to the syringe pump and barometer to set the barometric pressure, and the upper channel inlet is locked in the barrel to prevent air from entering the channel. Platinum wires are also located at the ends of both channels and are connected to the alternator and resistance meter through an alligator clip. When the intestinal organoid is located at the point where the first tube forming the primary channel and the second tube forming the multi-channel meet by gravity, a constant negative pressure (about −10 hpa) is set to absorb the intestinal organoid at the multi-channel entrance. When the set negative pressure is reached, the resistance value can be measured by flowing an AC frequency (about 50 Hz).

The present invention also provides a method for evaluating the barrier integrity of an intestinal organoid comprising the following steps:
step of filling an inside of the first tube of the impedance-based intestinal organoid evaluation system of claim 1 with a physiological saline or culture solution, and introducing the intestinal organoid into the first tube;
step of positioning the intestinal organoid to contact one end of the second tube, and measuring a first impedance using an impedance analyzer;
step of forming a negative pressure in the intestinal organoid deformation generating unit, and measuring a second impedance; and
step of evaluating the barrier integrity of the intestinal organoid from the first impedance and the second impedance

The step of evaluating the barrier integrity of the organoid can be to evaluate the difference between the resistance value obtained from the first impedance and the resistance value obtained from the second impedance. The TJ function of the organoid can be evaluated by the difference between the resistance value before the negative pressure is set and the resistance value when the negative pressure is stabilized.

In addition, the present invention provides a method for evaluating the barrier integrity of an intestinal organoid comprising the following steps:
step of filling an inside of the first tube of the impedance-based intestinal organoid evaluation system of claim 1 with a substance that induces membrane function damage and efficacy, and injecting an intestinal organoid into the first tube;
step of positioning the intestinal organoid to contact one end of the second tube, and measuring a first impedance using an impedance analyzer;
step of forming a negative pressure in the intestinal organoid deformation generating unit, and measuring a second impedance;
step of measuring a third impedance at regular time intervals in a state where the negative pressure is stable; and
step of evaluating the barrier integrity affected by the substance that induces membrane function damage and efficacy from the first impedance, the second impedance and the third impedance.

The step of evaluating the barrier integrity can be to evaluate the barrier integrity using a time t value when the value of equation 1 is 50% using the resistance value obtained from the first impedance (R₀), the resistance value obtained from the second impedance (R_{HIO}), and the resistance value obtained from the third impedance when time t has passed (Rₜ). When the resistance value at the time of organoid adsorption is R_{HIO}, when the resistance value at the time of organoid removal is R₀, and when time t is passed, the resistance value is Rₜ, the damage rate of the barrier integrity by the experimental drug can be measured at the time t value when the value of equation 1 is 50% by comparing the R_{HIO}−R₀ resistance value based on 100%.

$$Damaged ratio of barrier integrity ( \% ) = ( {R}_{t} - {R}_{o} ) / ( {R}_{HIO} - {R}_{o} ) \qquad < Equation 1 >$$

### Example

### 1. Intestinal Organoid Formation

A 35-mm tissue culture dish was applied with 1 ml of a coating solution with 5% matrigel diluted in DMEM-F12 medium, and then coated in a 37° C. incubator for 1 hour.

The cultured human pluripotent stem cell colony is cut into a size of 250×250 (μm) and then separated from the culture vessel by treating collagenase type IV.

The coating solution in the coated 35-mm tissue culture dish was removed, and the separated human pluripotent stem cells and mTeSR1 culture medium were added into the culture dish, followed by culture. During culture for 3 days, when the cell density of the cultured pluripotent stem cells became 70% or more of the total surface, definitive endoderm (DE) differentiation was induced.

In order to induce the differentiation of the cultured human pluripotent stem cells into a definitive endoderm, they were cultured in RPMI 1640 medium containing 0%, 0.2%, and 2% fetal bovine serum (FBS) and 100 ng/ml of Activin A for 3 days.

In order to differentiate the cells into a three-dimensional hindgut (HG) spheroid, 500 ng/ml of FGF4 and 3 μM CHIR99021 were added to DMEM-F12 medium containing 2% FBS, followed by culture for 4 days.

Then, the spontaneously generating three-dimensional hindgut spheroid was inserted into the matrigel dome, and three-dimensionally cultured in advanced DMEM-F12 medium containing 1× B27 supplement, 100 ng/ml of EGF, 100 ng/ml of Noggin and 500 ng/ml of R-spondin 1 to differentiate into a human intestinal organoid.

The formed human intestinal organoid (immature control, control HIO) was maintained by subculture once every 14 days with exchanging the medium once every 2 days. Additionally, the mature intestinal organoid (mature HIO) was cultured by subculture at least twice with treating with 1 ng/ml of IL-2.

### 2. Intestinal Organoid Maturation

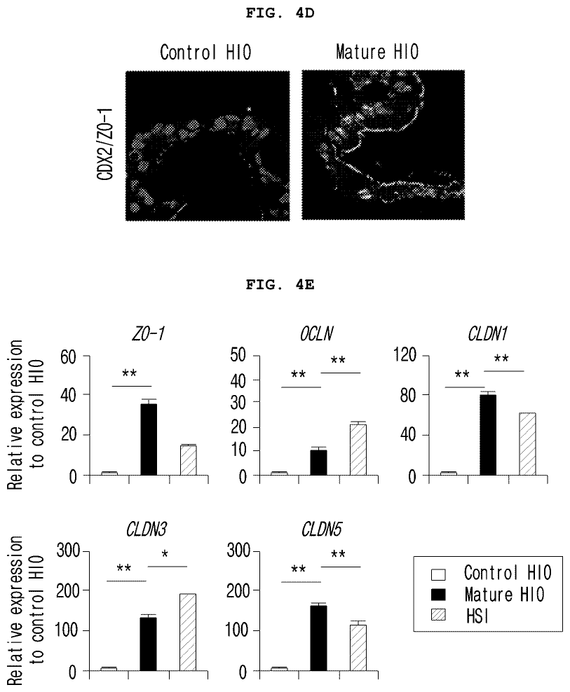

As shown in FIG. 4, the human pluripotent stem cells were differentiated into the intestinal organoid through the definitive endoderm and hindgut spheroid stages by the above-described intestinal organoid formation protocol, and it was confirmed that the intestinal organoid was efficiently differentiated by stage-specific morphological analysis.

In addition, when the expressions of marker genes of intestinal transcription factors (CDX2, SOX9, ISX), mesenchymal tissue (VIM), pinocytes (VIL1), enteroendocrine cells (CHGA), goblet cells (MUC2), and oncocytes (LYZ) were analyzed by qRT-PCR as the marker genes specifically expressed in the cells of each differentiation stage, it was confirmed that the expressions were significantly increased during differentiation of the intestinal organoid compared to undifferentiated pluripotent stem cells (FIG. 4a). For qRT-PCR analysis, human pluripotent stem cells, definitive endoderm cells, hindgut cells, control intestinal organoids, and mature intestinal organoids were collected, total RNA was extracted using a RNeasy kit, and cDNA was synthesized using Superscript IV First-Strand Synthesis System. Then, the analysis was performed using the primers targeting intestinal cell-specific marker genes and 7500 Fast Real-Time PCR System. Human small intestine total RNA (HSI) was purchased and used as a positive control. Morphological changes according to intestinal organoid maturation (FIG. 4b, upper panel) were investigated. As a result, it was confirmed that the size of the intestinal organoid (FIG. 4b, lower left panel) and the number of budding structures of the intestinal organoid (FIG. 4b, lower right panel) were increased in the mature intestinal organoid (Mature HIO) treated with IL-2 compared to the control intestinal organoid (Control HIO). The expression level of the mature intestinal marker gene was confirmed by qRT-PCR and compared with the control. As a result, it was confirmed that the expression of the mature intestinal organoid was increased similarly to that of the human small intestine (HSI) (Figure [symbol] 4c). As a result of immunostaining analysis, it was confirmed that the conjugated protein (ZO-1) was highly expressed in the mature intestinal organoid compared to the control intestinal organoid (FIG. 4d). For immunofluorescence staining analysis, the intestinal organoid was collected and fixed with 4% paraformaldehyde (PFA), then cryoprotected in 10%, 20% and 30% sucrose solutions, and then frozen using O.C.T compound. The frozen intestinal organoid sample was cut into 10 μm thick slices using a microtome, and permeated with a PBS solution containing 0.1% Triton X-100. After blocking in PBS containing 4% bovine serum albumin (BSA) for 1 hour, they were reacted with Anti-CDX2 antibody and Anti-ZO-1 antibody overnight at 4° C. After reacting with the secondary antibody at room temperature for 1 hour, the nuclei were stained with DAPI at room temperature for 15 minutes and observed through a confocal microscope. Gene expression of conjugated proteins (ZO-1, OCLN, CLDN1, CLDN3, and CLDN5) involved in membrane function was analyzed. As a result, it was confirmed that the conjugated protein genes were highly expressed in the mature intestinal organoid at a level corresponding to that of the human small intestine (HSI) compared to the control intestinal organoid (FIG. 4e). (*: p<0.05 control group vs experimental group according to t-test, **: p<0.01 control group vs experimental group according to t-test, ***: p<0.001 control group vs experimental group according to t-test)

### 3. Evaluation of Barrier Integrity of Intestinal Organoid

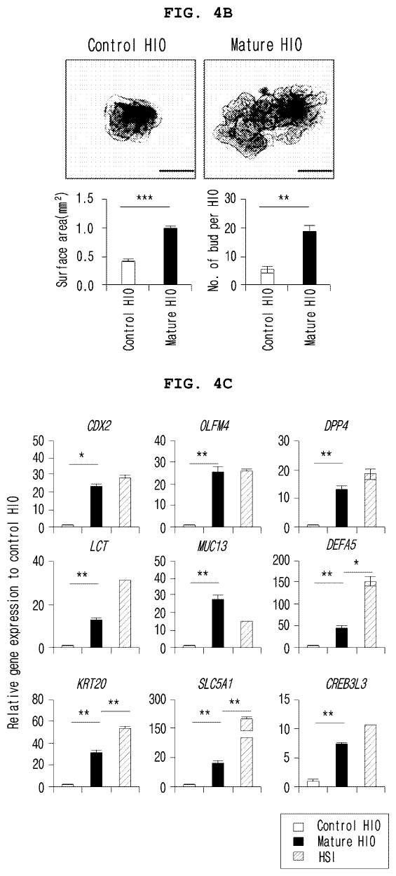

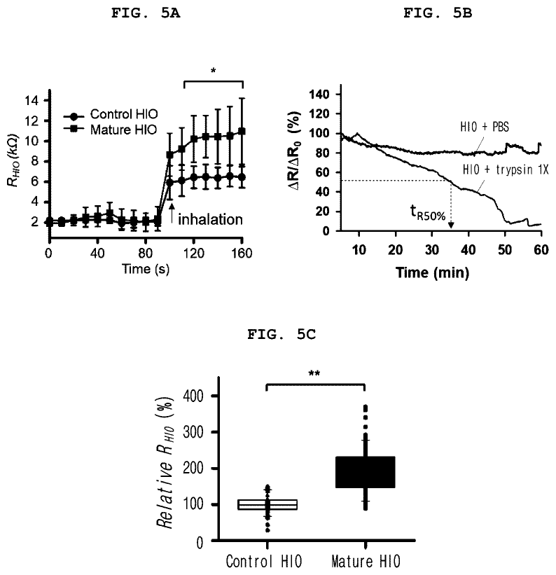

An impedance-based organoid evaluation system was configured as shown in FIG. 3 to evaluate the barrier integrity of the intestinal organoid.
1) Inside the organoid evaluation system, a channel having a structure in which a first tube having a diameter of 900 μm and a length of 4.5 mm and a plurality of second tubes for forming a multi-channel having a diameter of 200 μm and a length of 6 mm was vertically placed.
2) After preparing two Y-shaped tubes (third and forth tubes), a platinum wire having at least 99% purity with a diameter of 0.5 mm and a length of 5 cm was positioned in the form of a coil on one side of the Y-shaped tube, and the end of the wire was exposed to the outside of the Y-shaped tube and sealed to prevent air and water from leaking. The upper end of the Y-shaped tube was connected to a reservoir containing PBS (physiological saline) through a tube, and the lower end of the Y-shaped tube was connected to a syringe pump and a barometer through a tube, respectively.
3) The inside was filled with PBS.
4) The cultured intestinal organoid was injected at the end of the upper Y-shaped tube, and placed on the side where the first and second tubes are connected and the multi-channel starts.
5) The working electrode/sensing electrode and the reference/counter electrode of the impedance analyzer (potentiostat or impedance analyzer) were connected to the platinum wire protruding out of the Y-shaped tube.
6) The resistance values were measured at intervals of 10 seconds by applying an alternating current of 200 mV and 50 Hz.
7) The negative pressure (about −10 hpa) was set.
8) The experiment was terminated when the negative pressure was stabilized.
9) The barrier integrity of the intestinal organoid was evaluated by the difference between the resistance value before the negative pressure setting and the resistance value when the negative pressure was stabilized. The results are shown in FIG. 5.

### 4. Evaluation of Membrane Damage Resistance of Intestinal Organoid

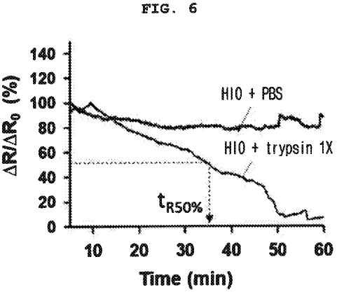

1) Inside the organoid evaluation system, a channel having a structure in which a first tube having a diameter of 900 μm and a length of 4.5 mm and a plurality of second tubes for forming a multi-channel having a diameter of 200 μm and a length of 6 mm was vertically placed.
2) After preparing two Y-shaped tubes (third and forth tubes), a platinum wire having at least 99% purity with a diameter of 0.5 mm and a length of 5 cm was positioned in the form of a coil on one side of the Y-shaped tube, and the end of the wire was exposed to the outside of the Y-shaped tube and sealed to prevent air and water from leaking. The upper end of the Y-shaped tube was connected to a reservoir containing membrane damage inducing drugs through a tube, and the lower end of the Y-shaped tube was connected to a syringe pump and a barometer through a tube, respectively.
3) The inside was filled with membrane damage inducing drugs.
4) The cultured intestinal organoid was injected at the end of the upper Y-shaped tube, and placed on the side where the first and second tubes are connected and the multi-channel starts.
5) The working electrode/sensing electrode and the reference/counter electrode of the impedance analyzer (potentiostat or impedance analyzer) were connected to the platinum wire protruding out of the Y-shaped tube.
6) The resistance values were measured at intervals of 10 seconds by applying an alternating current of 200 mV and 50 Hz.
7) The negative pressure (about −10 hpa) was set.
8) In a state where the negative pressure was stable, the resistance was measured every 10 seconds for 1 hour.
9) When the resistance value at the time of organoid adsorption is Rₕᵢₒ, when the resistance value at the time of organoid removal is R₀, and when time t is passed, the resistance value is Rₜ, the damage rate of the barrier integrity by the experimental drug was measured as the time t value when the value of Rₜ−R₀/Rₕᵢₒ−R₀ was 50% by comparing the Rₕᵢₒ−R₀ resistance value based on 100%. The results are shown in FIG. 6.

What is claimed is:

1. An impedance-based organoid evaluation system comprising:
an organoid deformation generating unit including a first tube; and a plurality of second tubes having a diameter smaller than that of the first tube, and being connected to one end of the first tube or inserted therein; and
an impedance measuring unit including an impedance analyzer for measuring an impedance of an organoid, the impedance analyzer being connected to the organoid deformation generating unit; and
wherein the impedance-based organoid evaluation system is configured to evaluate the organoid from the impedance measured by the impedance measuring unit.

2. The impedance-based organoid evaluation system according to claim 1, wherein the diameter of the second tube is 5% to 30% of the diameter of the first tube.

3. The impedance-based organoid evaluation system according to claim 1, wherein the organoid deformation generating unit comprises at least 3 second tubes.

4. The impedance-based organoid evaluation system according to claim 1, wherein the organoid deformation generating unit comprises:
a third tube connected to the other end of the first tube; and
a fourth tube connected to one end of the first tube or one end of the at least three second tubes.

5. The impedance-based organoid evaluation system according to claim 4, wherein the organoid evaluation system is connected to a reservoir through the third tube to introduce a material in the reservoir into the first tube.

6. The impedance-based organoid evaluation system according to claim 5, wherein the reservoir stores one of a physiological saline, a culture solution, a membrane function impairment inducing solution, and a membrane function efficacy inducing material.

7. The impedance-based organoid evaluation system according to claim 4, wherein the third tube includes an electrode material therein, and the electrode material is in a form of a coil-shaped wire.

8. The impedance-based organoid evaluation system according to claim 4, wherein the fourth tube is connected to a syringe pump.

9. The impedance-based organoid evaluation system according to claim 8, further comprising a pressure sensor connected to the syringe pump.

10. The impedance-based organoid evaluation system according to claim 8, wherein the fourth tube includes an electrode material therein, and the electrode material is in a form of a coil-shaped wire.

11. A method for evaluating the barrier integrity of an organoid comprising the following steps:
step of filling an inside of the first tube of the impedance-based organoid evaluation system of claim 1 with a physiological saline or culture solution, and introducing the organoid into the first tube;
step of positioning the organoid to contact one end of the second tube, and measuring a first impedance using an impedance analyzer;
step of forming a negative pressure in the organoid deformation generating unit, and measuring a second impedance; and
step of evaluating the barrier integrity of the organoid from the first impedance and the second impedance.

12. The method for evaluating the barrier integrity of an organoid according to claim 11, the step of evaluating the barrier integrity of the organoid is to evaluate using difference between a resistance value obtained from the first impedance and a resistance value obtained from the second impedance.

13. A method for evaluating the barrier integrity of an organoid comprising the following steps:
step of filling an inside of the first tube of the impedance-based organoid evaluation system of claim 1 with a substance that induces membrane function damage and efficacy, and injecting an organoid into the first tube;
step of positioning the organoid to contact one end of the second tube, and measuring a first impedance using an impedance analyzer;
step of forming a negative pressure in the organoid deformation generating unit, and measuring a second impedance;
step of measuring a third impedance at regular time intervals in a state where the negative pressure is stable; and
step of evaluating the barrier integrity affected by the substance that induces membrane function damage and efficacy from the first impedance, the second impedance and the third impedance.

14. The method for evaluating the barrier integrity of an organoid according to claim 13, the step of evaluating the barrier integrity is to evaluate the barrier integrity using a time t value when the value of equation 1 is 50% using the resistance value obtained from the first impedance (R₀), the resistance value obtained from the second impedance (Rₕᵢₒ), and the resistance value obtained from the third impedance when time t has passed (Rₜ).
$$Damaged ratio of barrier integrity ( \% ) = ( {R}_{t} - {R}_{o} ) / ( {R}_{HIO} - {R}_{o} ) \qquad < Equation 1 >$$
